(19) United States
(12) Patent Application Publication
(10) Pub. No.: US 2017/0151173 A1
(43) Pub. Date: Jun. 1, 2017

(54) COMPOSITIONS AND METHODS FOR OTOLOGIC PROPHYLAXIS AND TREATMENT

(71) Applicant: Otic Pharma Inc., Irvine, CA (US)

(72) Inventors: Gregory J. Flesher, Corona del Mar, CA (US); Catherine Crisp Turkel, Newport Coast, CA (US); Alan J. Mautone, Morristown, NJ (US); Sujana S. Chandrasekhar, Glen Ridge, NJ (US)

(73) Assignee: Otic Pharma Inc., Irvine, CA (US)

(21) Appl. No.: 15/365,321

(22) Filed: Nov. 30, 2016

Related U.S. Application Data
(60) Provisional application No. 62/260,732, filed on Nov. 30, 2015.
(60) Provisional application No. 62/260,734, filed on Nov. 30, 2015.
(60) Provisional application No. 62/260,742, filed on Nov. 30, 2015.

(51) Int. Cl. A61K 9/00; A61K 31/685

(52) U.S. Cl. CPC A61K 9/0043; A61K 31/685

(57) ABSTRACT

A prophylactic method for preventing otitis media or Eustachian tube dysfunction, or reducing the severity of symptoms thereof, decreasing the risk of future otitis media, with or without ear infection, and associated effects including acute or permanent hearing loss, speech and balance problems, behavior problems and sleep problems caused by acute, intermittent episodic, recurrent or chronic otitis media, and the need for surgical interventions involving myringotomy such as ear tubes for chronic middle ear effusions, or tympanic membrane perforation for acute drainage of effusion from the middle ear and ventilation of the middle ear, through the nasal delivery of surfactant to the Eustachian tube(s).

### BACKGROUND

Related Applications

This application claims the benefit of U.S. Provisional Application No. 62/260,732, filed Nov. 30, 2015, U.S. Provisional Application No. 62/260,734, filed Nov. 30, 2015, and U.S. Provisional Application No. 62/260,742, filed Nov. 30, 2015, which are incorporated by reference in their entirety.

Field

The present disclosure is a preventive or therapeutic agent for preventing Eustachian tube dysfunction (ETD) and preventing otitis media (OM). The present disclosure is further a preventive or therapeutic agent directed to compositions and methods of treatment for 1) restoring ear health and function, 2) reducing middle ear pressure, both positive and negative, or the sensation of middle ear pressure, to relieve discomfort or pain, 3) preventing hearing loss, 4) preventing acute, recurrent, or chronic OM, 5) preventing procedures such as placement of ventilation tubes in the tympanic membrane, or 6) preventing tympanic membrane perforation. The present disclosure also discloses a method that prevents or reduces the use of other medications such as antibiotics, steroids, analgesics, antihistamines, or decongestants in patients with ETD or OM. The present disclosure is further directed to post-surgical treatment methods and compositions, and more particularly, treatment methods and compositions for inhibiting ETD, improving healing time and improving outcomes post-surgery.

Description of Related Art

The mammalian ear is a complex and delicate organ comprised of three main parts: the outer ear, the middle ear and the inner ear. The outer ear collects sound and transfers it to the tympanic membrane causing it to vibrate. The tympanic membrane separates the outer and middle ear space. Vibrations of the tympanic membrane are conducted through the middle ear via small boney structures known as ossicles, which also vibrate in response to the tympanic membrane. The ossicles carry the sound waves through the middle ear to the inner ear where the cochlea translates the vibrations into nerve impulses, which the brain interprets as sound.

Each middle ear connects to the nasopharynx by means of the ET, which is responsible for ventilation, drainage, and protection of the enclosed middle ear space. The ET is mostly closed; it opens briefly (0.5 sec) during swallowing or yawning (˜1.5× per minute). An open ET equalizes atmospheric pressure in the middle ear. A closed ET protects the middle ear from unwanted pressure fluctuations and loud sounds. . The closure forces on the ETs are passive and include, for example, the adhesive force of the mucous blanket covering the mucosal surfaces, the elastic forces of adjacent supportive tissues, and the hydrostatic pressure of venous blood. The opening of the ETs is a result of the tensor veli palatini muscle, which is activated by swallowing and yawning. OME is ubiquitous in children who have a cleft palate, due to the lack of proper insertion of the tensor veli palatini muscle in the soft palate, rendering the muscle unable to open the Eustachian tube on swallowing or wide mouth opening. Most people who have flown on airplanes have had the experience of opening the ETs by yawning in order to equalize air pressure in the ear. The ETs, when closed, also serve to insulate the ears from sounds originating inside the head, such as speech, chewing, etc., as well as from sinus, nasal, or respiratory-borne pathogens injected into the nasopharynx during coughing. Due to inflammatory mechanisms, mucosal changes, sputum, and other factors, one or both ETs may become plugged or may not open periodically. This may be caused by increased force and pressure at the openings or on the walls and surfaces of the ETs, which increases the surface tension within the ETs. This condition is often referred to as ETD. When this occurs, sounds may be muffled and your ear may feel full, or you may have ear pain. The inability of the ETs to open naturally can prevent draining of the middle ear and cause fluid buildup, which can lead to infection or other morbidities. When the ET does not function normally and the middle ear becomes filled with fluid or creates a pressure differential with the ambient environment, the ear may lose its function as an auditory organ.

The ear, particularly in children, is a site of frequent OM episodes (inflammation in the middle ear sometimes with accompanying infections), which can be painful for the child, and distressing to parents of children not able to communicate. OM is frequently accompanied by ETD preventing the drainage of fluids from the middle ear and pressure equalization, which can result in negative middle ear pressure differential relative to the atmosphere. This negative pressure is not only uncomfortable and frequently painful; it can contribute to acute or permanent hearing loss, speech delay and/or balance problems. When the ET becomes does not function normally and the middle ear becomes filled with inflammatory fluid, the ear may lose its function as an auditory organ. In children, frequent episodes of OM can indicate the need for ear tubes, also known as tympanostomy or ventilation tubes. In both children and adults, severe OM may require a surgical procedure, called a myringotomy, in which a tiny incision is created in the eardrum (tympanic membrane) to relieve pressure caused by excessive buildup of fluid, or to drain the fluid from the middle ear.

OM or inflammation of the middle ear (comprising the middle ear cavity and ossicles) is an umbrella term that encapsulates acute OM (AOM) and OM with effusion (OME; ‘glue ear’). AOM is characterized by the presence of fluid in the middle ear together with signs and symptoms of an acute infection. OME is characterized by the presence of middle ear effusion behind an intact tympanic membrane; but, in contrast to AOM, OME is not associated with signs and symptoms of an acute infection. Recurrent OM (frequent, intermittent episodes of AOM and/or OME over months), as well as chronic OME (presence of fluid in the middle ear for at least 3 consecutive months) can result in damage to the tympanic membrane and ossicles in the middle ear which can result in temporary or permanent loss of hearing, perforations of the tympanic membrane, scarring, tinnitus, vertigo, and other serious morbidities. The American Academy of Pediatrics published a Clinical Practice Guideline “The Diagnosis and Management of Acute Otitis Media” in 2013, updating previous 2004 guidelines. Pediatrics 2013: Vol. 131; pp. e964 et seq. (March 2013). The guidelines acknowledge that “[a]ccurate diagnosis of AOM in infants and young children may be difficult,” citing the overlap of symptoms with upper respiratory tract infections, the obscuring of the TM by cerumen, as well as difficulties communicating with children. Id. at e972. The guidelines recommend, through a series of Key Action Statements (KAS), approaches to clinical treatment of patients presenting with possible AOM. AOM is affirmatively diagnosed when children present with moderate to severe bulging of the tympanic membrane (TM) or new onset of otorrhea (KAS 1A), or with mild bulging of the TM along with onset of ear pain within the past 48 hours or intense erythema (KAS 1B). The recommendations state that clinicians should not diagnose AOM in children who do not have middle ear effusion (KAS 1C). In sum, the 2013 Clinical Practice Guideline requires AOM to be diagnosed only after the disorder has manifested in moderate to severe TM bulging, otorrhea or effusion. Before this stage is reached, clinicians are counseled to consider observation only, without the use of antibiotics, to see if the condition resolves spontaneously. The prophylactic use of antibiotics is discouraged, because the symptoms may be of viral etiology, the antibiotics might induce avoidable side effects, and because of the risk of proliferation of antibiotic-resistant strains of various pathogens.

Unfortunately, many children are subject to eventual development of AOM meeting the Pediatrics Clinical Practice Guideline before being administered antibiotics—the standard of care that provides relief only when bacterial infection is the etiology of the AOM, and even then requires days of therapy to result in relief from some of the more oppressive symptoms of AOM. Other impacts of AOM include school absences, parent days missing work, etc.

An updated Clinical Practice Guideline was issued in 2016 on Otitis Media with Effusion (OME) from the American Academy of Otolaryngology-Head and Neck Surgery. Otolaryngology-Head and Neck Surgery, 2016, Vol. 145 (IS), pp. S1-S41. The guidelines recommend against using intranasal or systemic steroids, systemic antibiotics, antihistamines, or decongestants for treating OME. The Guideline discusses the healthcare burden posed by OM, stating that outpatient visits to pediatrician's accounts for 11.4% office encounters in primary care practices. Of these OM visits, about 1 in 3 are for OME, which can present as the primary diagnosis (17%), or in conjunction with AOM (6.5%). At least 25% of OME episodes persist for 3 months and may be associated with hearing loss, balance (vestibular) problems, poor school performance, behavioral problems, ear discomfort, recurrent AOM, or reduced quality of life (QOL). OME is described as the most common cause of hearing impairment in children, and permanent hearing loss related to OM has a prevalence of 2 to 35 per 10,000. Id. at 55, 59.

Middle ear effusion in at least one ear early after birth 48 hrs.) has been reported to occur in 7-11% of all neonates. Chang K. W. et al., Otolaryngol. Head Neck Surg., 1993; Doyle K. J. et al., Otolaryngol. Head Neck Surg., 1997; Doyle K. J. et al., Otology & Neurotology, 2004. OME accounts for about two-thirds of newborn hearing screening failures. Schilder A. G. M., et al., Nature Reviews 2016. MEE present at 30-48 hours after birth is a risk factor for developing COME [≧3 consecutive months with MEE]. Doyle K. J. et al., Otology & Neurotology, 2004, 58% neonates w/MEE vs. 20% of neonates without MEE at 30-48 hours old developed COME within first year. Id. Suppurtative middle ear effusions have been shown to occur commonly in neonates. Balkany et al., “Middle ear effusions in neonates,” Laryngoscope, 1978 Mar; 88(3):398-405.

In children with uncomplicated non-severe AOM who are not at increased risk of complications, watchful waiting or delayed antibiotic prescription (only filed when symptoms of AOM persist for 48-72 hours) is recommended. Antibiotics are not recommended for treatment of OME. However, many patients will demand, and many clinicians will prescribe, antibiotics. Even though antibiotics will have no effect on a viral infection or accumulation of nonpurulent fluid in the ear due to ETD, most parents and patients feel the need to “do something” rather than wait for a condition to spontaneously resolve. As well, if the accumulation of fluid is due to a bacterial infection, the earlier a course of antibiotics is commenced, the generally earlier-resolved and less uncomfortable the infection. However, recent studies have concluded that there is a problem with the over-prescription of antibiotics, which is contributing to the appearance and evolution of antibiotic-resistant “super bugs” that could present major healthcare challenges in the coming years. Many public health authorities have proposed the issuance of antibiotic prescription guidelines to lower the overall rate of prescription and use of antibiotics.

A recent report cites to the lack of consensus on the definition and diagnosis of ETD, and proposing a definition, clinical presentation and diagnosis of ETD. “ET dysfunction: consensus statement on definition, types, clinical presentation and diagnosis,” Clinical Otolaryngology, 40 (2015): 407-411. The paper begins by pointing out the complex function of the ET, but settles on three principle roles: 1) pressure equalization and ventilation of the middle ear; 2) mucociliary clearance of secretions from the middle ear; and 3) protection of the middle ear from sounds, and from pathogens and secretions from the nasopharynx. Id. at 407. The paper recognizes the existence of baro-challenge ETD, which occurs only on change in ambient pressure, as a distinct subtype of ETD. The recommendations go on to state, however, that “ET dysfunction should not be used to describe disease more properly classified as otitis media, including chronic otitis media with effusion (glue ear), chronic suppurtative otitis media, tympanic membrane retraction, and cholesteatoma.” Id. at 410.

Compositions have been proposed to improve the flow of both naturally occurring fluids and pharmacologic agents through the mammalian ET. For example, U.S. Pat. No. 6,676,930 to Alan J. Mautone discusses a composition comprised of a surfactant and a spreading agent, as well as a therapeutic agent, delivered nasally to the ET to maintain its patency. The patent discloses symptomatic treatment of OM by a non-toxic, non-antibiotic nasally administered composition.

WO 97/29738 describes inhaling surfactants to maintain the opening pressure of the ET. The disclosure is based on experiments involving securing a cannula through the perforated tympanic membrane of a gerbil before filling the outer ear with epoxy.

Steroid nasal sprays have also been developed to try and help with ETD. Some steroid nasal sprays require a Valsalva maneuver be performed in order to deliver the steroids to reach the ETs. As known in the art, the Valsalva maneuver involves blowing air out the nose while keeping the nares pinched in order to force the ETs open and introduce nasal spray medicine up into the ETs. This is an active maneuver that is often prescribed to be done regularly throughout the day during treatment. Although, the Valsalva maneuver may be helpful in some cases of administering steroid nasal sprays, it is less than optimal for a number of reasons. First, attempting to blow—air from the nose—when congested might be impossible. Second, if the Valsalva maneuver does work in opening the ETs, it only lasts for a second or two, thus there is no sustained efficacy over time. Third, the momentary opening allows a bit of pressure relief and equilibrium, but it does not allow drainage of fluid from the middle ear. Lastly, the Valsalva maneuver is not a viable option for use with infant and young children due to the series of active actions required by the patient.

### SUMMARY

One aspect of the present disclosure is directed to the use of surfactant compositions for otologic prophylaxis to reduce the risk or frequency of future OM episodes, with or without ear infection, restore hearing loss caused by middle ear fluid, improve speech problems and balance problems, improve behavior and sleep problems caused by chronic OM, and prevent the need for surgical interventions involving insertion of ventilation tubes into the tympanic membrane for OM or need to perform a myringotomy (tympanic membrane perforation for acute drainage and ventilation of the middle ear.

Another aspect of the present disclosure is directed to the use of surfactant compositions for otologic prophylaxis to reduce the risk or frequency of extracranial complications associated with chronic OM, wherein the extracranial complication includes at least one of facial nerve paresis, labyrinthitis, mastoiditis, and petrositis.

Another aspect of the present disclosure is directed to the use of surfactant compositions for otologic prophylaxis to reduce the risk or frequency of intracranial complications associated with recurrent or chronic OM, wherein the intracranial complications include at least one of brain abscess, epidural abscess, meningitis, lateral sinus thrombosis, subdural cavernous thrombosis, subdural abscess, cerebellitis, labyrinth sclerosis.

Another aspect of the present disclosure is directed to the use of surfactant compositions for otologic prophylaxis to reduce the risk or frequency of cholesteatoma associated with recurrent or chronic OM.

Another aspect of the present disclosure is directed to the use of surfactant compositions for otologic prophylaxis to prevent future damage to a patient's ear including scarring to the middle ear bones, damage to the eardrum, and damage to the inner ear.

Another aspect of the present disclosure is directed to the use of surfactant compositions for otologic prophylaxis to prevent or reduce the frequency of tinnitus, dizziness, vertigo, nausea, or facial pain.

Another aspect of the present disclosure relates to a method for nasal delivery of a surfactant to a patient which comprises: an aerosolized amount of surfactant delivered through the patient's nostrils; and optionally repeating the deliver step a sufficient number of times until an effective amount of surfactant is delivered to the ETs of a patient. The method may also include optionally repeating the administering step a sufficient number of times over a short (e.g., minutes, hours or days) or long (e.g., days, weeks to months) period of time until an effective amount of surfactant is delivered to the patient and/or the patient's ET function is restored. The administering step can be performed acutely, chronically or intermittent-chronically.

Another aspect of the present disclosure is directed to a method of restoring ear health by treating and reducing the frequency and severity of OM with or without ear infection, fever, ear pain, hearing loss, irritability, sleeping problems, ear discharge, speech and balance problems, and nausea and vomiting.

Another aspect of the present disclosure is directed to a method of restoring ear health by treating and reducing the severity of extracranial complications associated with recurrent or chronic OM, wherein the extracranial complications include at least one of facial nerve paresis, labyrinthitis, mastoiditis, and petrositis.

Another aspect of the present disclosure is directed to a method of restoring ear health and reducing the severity of intracranial complications associated with recurrent or chronic OM, wherein the intracranial complications include at least one of brain abscess, epidural abscess, meningitis, lateral sinus thrombosis, subdural cavernous thrombosis, subdural abscess, cerebellitis, labyrinth sclerosis.

Another aspect of the present disclosure is directed to a method of restoring ear health by treating and reducing the severity of cholesteatoma associated with recurrent or chronic OM.

Another aspect of the present disclosure is directed to a method of restoring ear health by treating and reducing the severity ear damage including scarring to the middle ear bones, damage to the eardrum, and damage to the inner ear.

These methods may include having the patient deliver a surfactant through the patient's nostrils, and optionally repeating the delivery step a sufficient number of times until an effective amount of surfactant is delivered to the ETs of a patient. In a patient with normal anatomy, each nostril accesses an ET: one accessed by the left nostril connecting to the left ear, and one accessed by the right nostril connecting to the right ear.

In some embodiments, the method may also include determining whether the patient's increased risk exceeds a risk threshold before administering the surfactant. In some embodiments, the surgery performed may be at least one of a myringoplasty, a tympanoplasty, an ossiculoplasty, a stapedectomy, a tympaneocentesis, or a myringotomy. In some embodiments, the method may also include prescribing a dose frequency based on the one or more factors. In some embodiments, at least one of the factors is the type of surgery the patient underwent. In some embodiments, at least one of the factors may be at least one of the patient's age, weight, allergies, and medical history. In some embodiments, at least one of the factors may be whether the patient is a smoker. In some embodiments, at least one of the factors may be the patient's planned activities, wherein the planned activity is at least one of airplane travel, scuba diving, and driving up or down mountains or other conditions in which there is a change in atmospheric pressure. In some embodiments, at least one of the factors may be whether the patient has a compromised immune system or immunodeficiency thus making them more susceptible to get a cold or the flu. In some embodiments, the mixture may be administered from a metered dose inhalation device filled with the surfactant. In some embodiments, the method may also include reducing the healing time of the patient post-surgery. In some embodiments, wherein the middle ear space becomes stabilized over time.

### BRIEF DESCRIPTION OF DRAWING(S)

FIG. 1 is a schematic illustration of the external, middle, and inner ear of a human being.

FIG. 2 is a schematic cross-sectional side view of a human head.

FIG. 3 is a flow chart of a treatment method, according to an exemplary embodiment.

FIG. 4 is a flow chart of a treatment method, according to a further exemplary embodiment.

### DETAILED DESCRIPTION

When “surfactant” is referred to it is understood that such a term refers to biocompatible surfactants generally, alone or with spreading agents. Fully saturated diacylphospholipids such as dipalmitoylphosphatidylcholine (DPPC) are native to the mammalian lung's epithelial lining. In addition to DPPC, naturally occurring spreading agents helping to uniformly spread surfactant system over the surfaces of the lung, including cholesteryl esters such as cholesteryl palmitate (CP), phospholipids such as diacylophosphatidylglycerols (PG), diacylphosphatidylethanolamines (PE), diacylphosphatidylserines (PS), diacylphosphatidylinositols (PI), sphingomelin (Sph) and Cardiolipin (Card); and virtually and other phospholipid, and of the lysophospholipids; or any of the plasmalogens, dialklylphospholipids, phosphonolipids, carbohydrates and proteins, such as, for example, albumin, pulmonary surfactant proteins A, B, C and D. The naturally occurring surfactant system is further described in U.S. Pat. No. 5,306,483.

The term “nasal delivery” refers to focal delivery of surfactant to a patient's ETs that are located in the nasopharynx through and into the nose. Focal delivery of the composition is not achieved via delivery into the nose towards the nasal cavity (i.e., upwards towards the forehead, like conventional nasal inhalers). The product is delivered towards the back of the nose to the nasopharynx area where the ETs are located. The goal is not a systemic treatment, but a local treatment to the ET.

The term “patient” refers to a mammal, particularly a human. A patient may be, for example, a subject that developed or is at risk of developing certain pathologies of the ear and surrounding tissue. A specific need arises in humans who are either suffering from, or likely to suffer from, tinnitus, vertigo, facial nerve pain, tonsillitis, headache, asthma, fever, upper respiratory tract infection, or tonsillopharyngitis, or exhibits symptoms including one or more of fever, ear pain, dulled hearing, irritability, difficulty sleeping, tugging of the ears, discharge (yellow, clear, bloody), loss of balance, dizziness, nausea, vomiting, diarrhea, decreased appetite or congestion.

The term “about” or “approximately” as used herein means within an acceptable error range for the particular value as determined by one of ordinary skill in the art, which will depend in part on how the value is measured or determined, e.g., the limitations of the measurements system. For example, “about” can mean within one or more than one standard deviation per the practice in the art. Alternatively, “about” can mean a range of up to 20%, such as up to 10%, up to 5%, and up to 1% of a given value.

The use of surfactants to treat a patient is well described in U.S. Pat. Nos. 6,156,294, 6,521,213, 6,616,913, 6,676,930, and 7,064,132, the disclosures of each are incorporated herein by reference.

As used herein, the term “effective amount” means an amount of surfactant which is capable of preventing, reducing the frequency of, or treating the various pathological conditions herein described, e.g., acute, recurrent and chronic OM, the sensation of middle ear pressure, ear pain, hearing loss, recurrent middle ear infection, scarring or erosion to the middle ear bones, damage to the eardrum, damage to inner ear components (E.g., cochlea, auditory nerve, etc.), or surgical intervention involving myringotomy to insert ventilation tubes into the tympanic membrane for recurrent or chronic ear infections, or myringotomy to perforate the tympanic membrane for acute drainage and ventilation of the middle ear.

The terms “inhibit” and “inhibiting” bear their usual meaning which includes prohibiting, treating, alleviating, ameliorating, halting, restraining, slowing or reversing the progression, or reducing the frequency or severity of a pathological condition described above. As such, these methods include both medical therapeutic (acute) and/or prophylactic (prevention) and/or ongoing (chronic) administration as appropriate.

The term “pharmaceutical”, when used as an adjective herein, means substantially non-toxic to the patient.

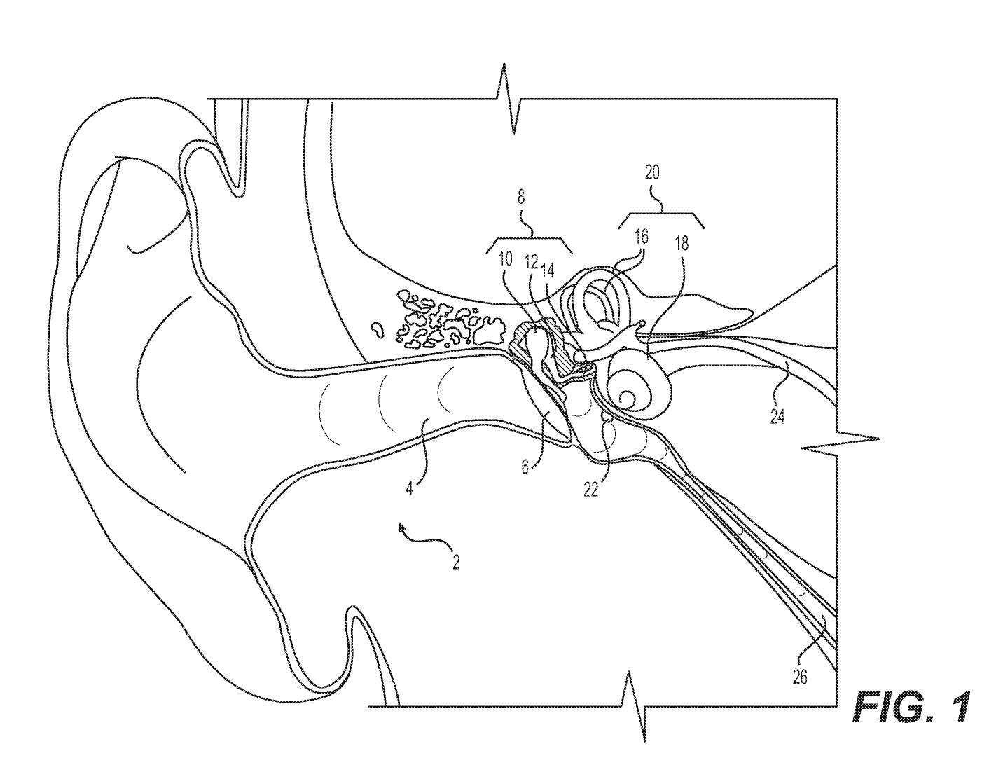

The human ear, shown generally in FIG. 1 is a complex organ comprising external, middle and inner portions. External ear 2 comprises the visible ear as well as the external auditory tube 4, where sound waves enter. The tympanic membrane 6, forming the “ear drum” is made of a thin sheet of skin-like material and defines a sealed tympanic cavity or middle ear 8. The middle ear is a small enclosed, air-filled cavity that needs to be protected and maintained in a state of homeostasis with the external environment. If fluid, (e.g. inflammatory media) gets into the middle ear or a pressure differential exists with the internal environment, then the sound waves cannot efficiently be conducted from tympanic membrane to the boney structures and ultimately to the inner ear, a condition known as conductive hearing loss. The middle ear 8 includes tiny bones, or ossicles, known as the malleus (or hammer) 10, the incus (or anvil) 12 and the stapes (or stirrup) 14. These ossicles cooperate to translate vibrations in the tympanic membrane 6 to the cochlea 18, which in turn converts mechanical vibrations to electrochemical signals via the auditory nerve for processing by the brain. Three semicircular canals including the anterior, lateral and posterior (not shown) form a labyrinth 16, which forms an important part of the vestibular system. Cochlea 18 is filled with an aqueous fluid called perilymph, which moves in response to the vibrations coming from the middle ear via round window 22. As the fluid moves, hair cells convert motion to electrical signals. The inner ear 20 comprises the labyrinth 16 and the cochlea 18. Cochlear or Auditory Nerve 24 conducts signals to the brain for processing.

The ET 26 connects the middle ear 8 to the pharynx. The ET allows the sealed tympanic chamber to equalize pressure with the ambient via the oronasal chambers, allows draining of fluid from the middle ear, and also serves to insulate the middle ear from sounds originating within the head, such as voice and mastication. The ET plays a key role in the normal function and health of the middle ear. ETs are compliant, liquid-lined tubes that: a) ventilate and maintain ambient air pressure in the middle ear; b) remove fluid from the middle ear; and c) prevent foreign material from entering the middle ear space. A normally functioning ET remains collapsed at the nasopharynx end of the tube, but opens periodically upon swallowing and yawning. Inflammation of the nasopharynx region due to factors such as upper respiratory infections (bacterial or viral), allergies, and other mediators of inflammation prevent normal ET function resulting in middle ear afflictions such as OM.

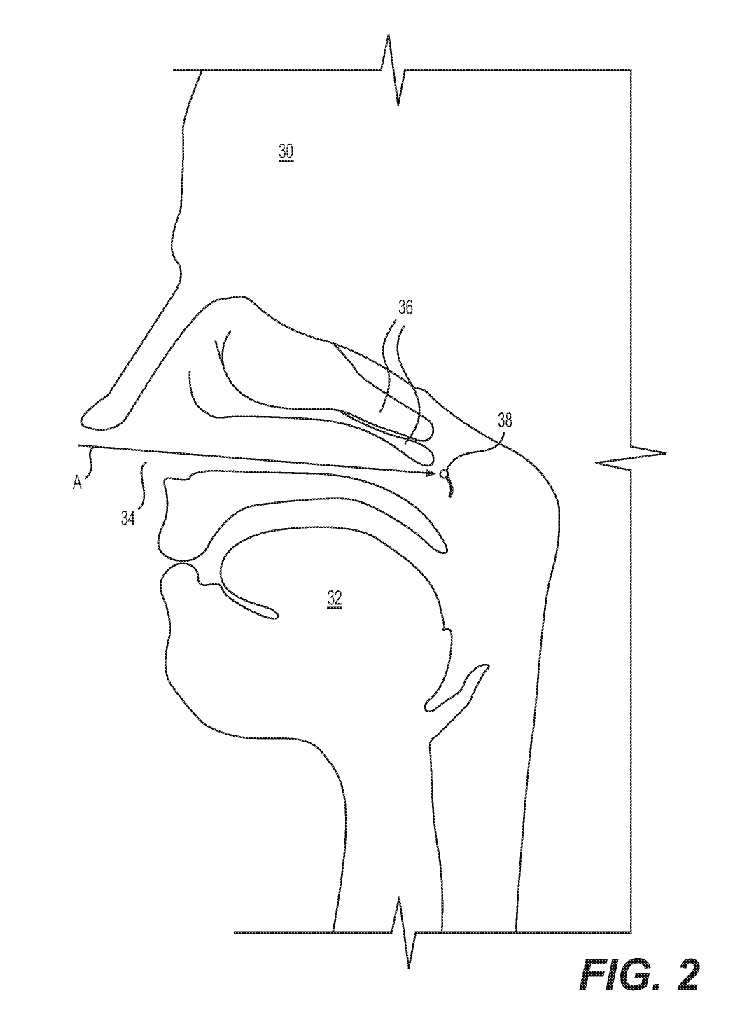

Turning to FIG. 2, human head 30 is depicted in schematic cross-section. Tongue 32 appears in the oral cavity, and nostril 34 opens to the nasal cavity containing nasal turbinates 36. At the posterior of the nasal cavity the ET openings 38 are located. Administration of surfactant via the nasal cavity, as discussed more fully below, can, in an illustrative embodiment, be achieved in the direction represented by arrow A. Administration of the surfactant according to the methods of the present disclosure may be passive, such that no Valsalva maneuver is required in order to deliver the surfactant to the ETs. Thus, the methods of the present disclosure provide a more consistent and simple approach that may be utilized with infants and young children. In addition, the methods of the present disclosure provide a sustained effect in contrast to the temporary pressure relief provided by the Valsalva maneuver.

The present disclosure relates to methods for the nasal delivery of surfactant to a mammalian patient, such as a human patient, and pharmaceutical formulations adapted for said nasal delivery. Some methods of the present disclosure may be carried out by dispersing an amount of surfactant in a volume of gas to produce an aerosolized amount. The dispersion may be produced, for example, by the patient's inspiratory breath (inhalation), by introducing a dry powder of lyophilized surfactant into a high velocity gas stream, by nebulizing or spraying a liquid solution or suspension of surfactant, or by releasing a propellant entraining or otherwise carrying surfactant through a nozzle. The surfactant is projected onto the region of the nasopharynx proximate to the opening(s) of the ET(s), and thereby permitted to enter into the ET(s). By repeating the dispersing steps a sufficient number of times, a desired total dosage (an effective amount) of surfactant can be delivered to the patient.

Some methods of the present disclosure may include dispersing a dose of surfactant in a volume of gas to produce and aerosolized amount and dispersing a dose of surfactant as a dry powder suspension formulation.

The present disclosure also relates to post-surgical treatment of a patient by the nasal delivery of a surfactant to inhibit one or more otologic conditions including, for example, ETD. The present disclose also relates to the pharmaceutical formulations adapted for said nasal delivery treatment. ETD may cause or be associated with a variety of other conditions or infections of the ear. For example, ETD may be associated with OM or OME. As described herein, ETD may refer to any situation where one or both of the ETs are not opening and closing naturally. For example, natural opening and closing of the ETs may be restricted when the ETs become inflamed or there is increased pressure on the walls of the ETs.

Surfactant may be made by established procedures, such as those detailed in U.S. Pat. No. 5,174,988, the teachings of which are herein incorporated by reference. Particular formulations of surfactant suitable for nasal delivery to a patient include dry powders, liquid solutions or suspensions suitable for nebulization or spraying, and propellant formulations suitable for use in metered dose inhalers (MDI's). The preparation of such formulations is well described in the patent, scientific, and medical literatures.

Dry powder formulations in illustrative embodiments include surfactants in a dry particulate form, which can be lyophilized. Particles can be formulated to an appropriate particle size or within an appropriate particle size range. Particle size appropriate for deposition within the nose can be about 0.5 μm mass median equivalent aerodynamic diameter (MMEAD), but in an exemplary embodiment can be about 3 μm MMEAD, and in a further exemplary embodiment can be about 5 μm MMEAD. Maximum particle size for deposition within the nose can be about 100 μm MMEAD, and in an exemplary embodiment is about 50 μm MMEAD, and in another exemplary embodiment is about 20 μm MMEAD, and in another exemplary embodiment is about 16 μm MMEAD and in a further exemplary embodiment is about 4 μm MMEAD. Compositions of surfactant can be obtained via numerous processes, e.g., crystallization, jet milling, spray drying, solvent precipitation, supercritical fluid condensation, homogenization, etc. In some embodiments, particles may be obtained via crystallization from solution and then subjected to further processing as needed.

Dry powder or crystallized formulations of surfactants can be administered using conventional metered dose inhalers (MDI) or conventional dry powder inhalers (DPI). Other methods known in the art for delivering medicaments nasally are also usable, with appropriate account taken of the fact that delivery is not intended to the lungs of the patient, but rather dispersion at or near the ET openings. Nozzles, tubes and other apparatus can be employed, and the surfactant can be delivered by any appropriate source of pressure, or by negative pressure created in the nasal cavity through inhalation.

In some embodiments, the dry powder devices require a dry inhalant mass, for example in the range from about 1 mg to 20 mg, to produce an aerosolized dose. In some embodiments, a dry bulking agent is added to the surfactant to make up the required mass. Exemplary dry bulking agents include dextrose, dextrans, trehalose, human serum albumin (HSA), sucrose, sodium ascorbate, mannitol, maltotriose, cellobiose, lactose, starch, pectin, sodium citrate, sodium ascorbate, glycine and the like.

Liquid formulations of surfactant are also envisioned for use in nebulizers which in illustrative embodiments can employ surfactant dissolved or suspended in a pharmaceutically acceptable solvent, e.g., water, ethanol, or mixtures thereof. The concentration of surfactant dissolved/suspended may vary, for example, between about 25-35 mg/ml, but in an exemplary embodiment is 13.5 mg/mL, and in another exemplary embodiment is 80 mg/m L. Some exemplary surfactants include, for example, Survanta®, Infasurf®, Exosurf®, and Curosurf®. The total volume of nebulized liquid needed to deliver an aerosolized amount of 0.1 mL is about 0.125 mL.

Other adjutants are also used in some exemplary embodiments, including preservatives, additional surfactants, antioxidants, or other stabilizing excipients and dispersants. Suitable preservatives include, but are not limited to benzalkonium chloride, phenylethyl alcohol and the like. Suitable further surfactants include, but are not limited to Acyl chain phospholipids, oleic acid, sorbitan trioleate, polysorbates, lecithin, phosphotidyl cholines, and various long chain diglycerides and other phospholipids. Suitable dispersants include, but are not limited to, ethylenediaminetetraacetic acid (EDTA), gasses, and the like. Suitable gases can also be mixed or used to propel or disperse the surfactant, including in exemplary embodiments air, nitrogen, helium, hydrofluoroalkane (HFA), carbon dioxide, air, and the like.

For use in MDI's, surfactant may be dissolved or suspended in a suitable aerosol propellant, such as a hydrofluoroalkane (HFA) or a hydrofluorocarbon (HFC). Such suspensions will contain between 0.5 mg to 100 mg of surfactant per aerosol dose in an illustrative embodiment, and in another embodiment from 1 mg to 5.4 mg, and in another embodiment from 2 mg to 4 mg. Suitable propellants are well known in the art. For incorporation into the aerosol propellant, surfactant can in illustrative embodiments be of the sizes described above for the dry powder or crystallized formulations. The particles may then be suspended in the propellant as is, or can be further coated in additional surfactants, particularly any additional components such as active ingredients added to the surfactant. Suitable additional surfactants are as defined above in the liquid formulation illustrative embodiment. As is known in the art, propellant formulations may further include additives such as an alcohol such as ethanol and/or other additives to aid formulation stability and physiological acceptability including additional surfactants as described above.

The precise dosage of surfactant necessary will vary with the age, size, sex and condition of the subject, the nature and severity of the condition to be treated, the course of prophylaxis or treatment and the severity of the risk of complication, and the like; thus, a precise effective amount should be determined by the caregiver.

In the case of a dry powder or crystalline formulation or a liquid formulation suitable for use in a nebulizer or other spraying appliance, the total dosage of surfactant can be administered in a single delivery cycle, or with a plurality.

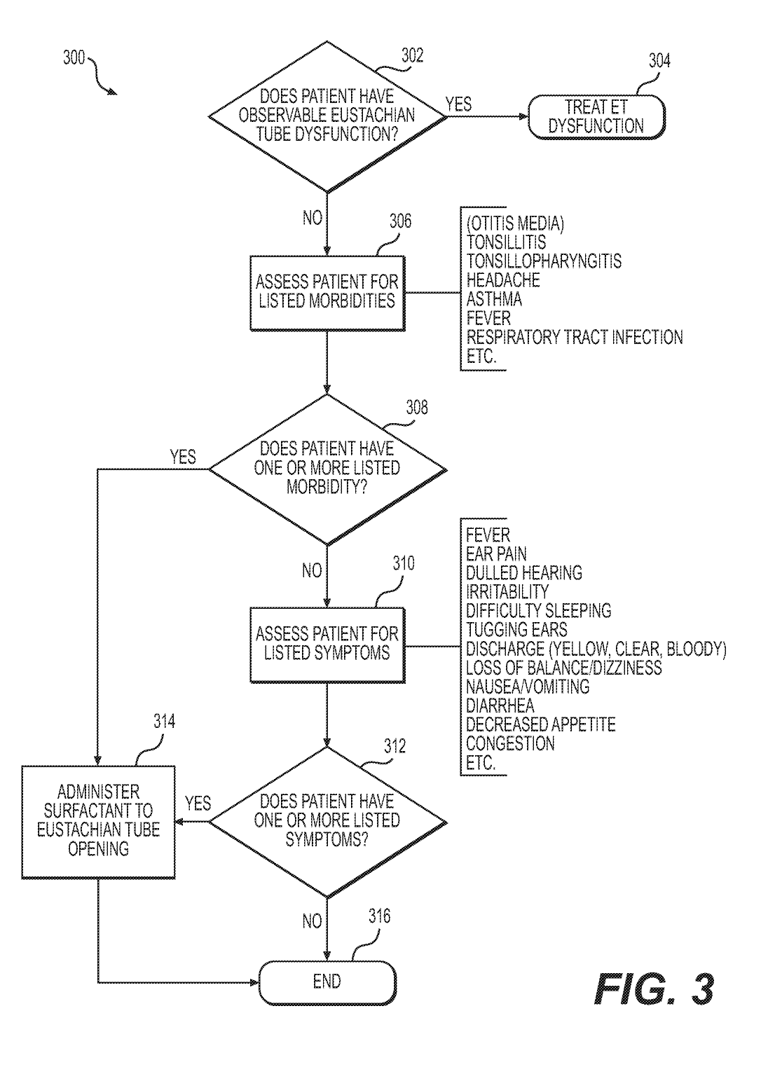

Turning to FIG. 3, a process for prophylactic treatment 300 is described. This process can also be employed for restoring ear health and reducing the severity of symptoms experienced by a patient. The patient presenting can first be assessed for active ETD at step 302, although clinically this is usually not done in current practice. The presence of ET dysfunction can be determined subjectively through a history of patient symptoms by a clinician, or objectively. Conventional tympanometry is a typical approach to diagnosis, where a tympanogram exceeding −250 decapascals (daPa) middle ear pressure is suggestive of ET dysfunction. This measure is not, however, conclusive. Active AOM, which suggests ET dysfunction, can be diagnosed by otoscopy and can be further assessed using a symptom severity scale. Pneumatic otoscopy is the primary diagnostic modality for OME, with otomicroscopy and tympanometry as adjunct measures. Otoscopy alone, without a pneumatic bulb, might overlook OME because the tympanic membrane might appear normal and ear-related symptoms can be minimal or absent. Tympanometry objectively measures tympanic membrane mobility and middle ear function. Compared with pneumatic otoscopy, tympanometry has comparable sensitivity (range: 90-94%) but lower specificity (50-75% versus 80% for tympanometry and pneumatic otoscopy, respectively) for diagnosing OME. See Schilder et al, 2016. Clinicians also examine the tympanic membrane for perforation, and perform additional measurements while the patient swallows, and while performing the Toynbee (hold nose and swallow) procedure and Valsalva maneuver (hold nose and blow) procedure and watch for pressure equalization. Perforated eardrums are tested through the application of direct pressure which dissipates rapidly if the ET is functioning. If there is clear evidence of ET dysfunction present, then that condition should be treated at step 304.

If there is no currently observable evidence of ET dysfunction, or if a diagnosis of ET dysfunction is not achievable (Step 302: No), then the patient may be assessed at step 306 for morbidities that could include OM, or lead to future OM, with or without ear infection, acute or permanent hearing loss, speech and balance problems, behavior problems and sleep problems caused by chronic ear infections, and the need for surgical interventions involving myringotomy such as ear tubes for chronic ear infections or tympanic membrane perforation for acute drainage and ventilation of the middle ear. These morbidities may include, for example, tinnitus, vertigo, facial nerve pain, tonsillitis, headache, asthma, fever, respiratory tract infection and/or tonsillopharyngitis. These morbidities can also include a recent history of one or more of the aforementioned morbidities, or a clinically meaningful recent history of ET dysfunction or middle ear maladies such as AOM, OME, etc. At step 308, it may be determined whether the patient has, or has recently had during a clinically relevant time period, one or more of the listed morbidities. Patients having, or having had, one or more of these conditions are administered surfactant to the ET opening, as described in this disclosure, at step 314. This administration can be prophylactic in nature, even where there is no current evidence of ET dysfunction or OM, in which case the listing of morbidities would likely exclude OM. OTITIS MEDIA is represented in parentheses in FIG. 3 to reflect this.

If these morbidities are absent at step 308, then the patient may be assessed at step 310 for symptoms that that could lead to, or may be associated with, future OM with or without ear infection, recurrent or chronic OM, acute or permanent hearing loss, speech and balance problems, behavior problems and sleep problems caused by chronic ear infections, and the need for surgical interventions involving myringotomy such as ear tubes for chronic ear infections or tympanic membrane perforation for acute drainage and ventilation of the middle ear. These symptoms may include, for example, fluid in the middle ear, fever, ear pain, dulled hearing, irritability, difficulty sleeping, tugging ears (particularly by pre-speech children), ear discharge (yellow, clear, or bloody), loss of balance and/or dizziness, nausea and/or vomiting, diarrhea, decreased appetite, and congestion. In the case of a neonate, the symptoms can include fluid present in the middle ear during the early life, for example the first month of life. After the patient is assessed at step 310 for symptoms, it may be determined at step 312 whether the patient has one or more of the above listed symptoms, or a recent history of these symptoms. Patients having one or more of these symptoms (Step 312: Yes) administered surfactant to the ET opening, as described in this disclosure, at step 314. The administration, as above, may be prophylactic.

If these symptoms are absent at (Step 312: No), the process ends at step 316, where the patient is kept under observation.

Other methods of treatment similar to treatment 300 are also disclosed herein. For example, a treatment method for decreasing the risk of, or restoring ear health by treating extracranial complications associated with recurrent or chronic OM is provided. The extracranial complications may include, for example, facial nerve paresis, labyrinthitis, mastoiditis, and petrositis. Another example treatment method may be for decreasing the risk of, or for treating, intracranial complications associated with chronic OM. The intracranial complications may include, for example, brain abscess, epidural abscess, meningitis, lateral sinus thrombosis, subdural cavernous thrombosis, subdural abscess, cerebellitis, labyrinth sclerosis. Yet another example treatment method may be for decreasing the risk of, or for restoring ear health by treating, cholesteatoma associated with recurrent or chronic OM. These treatment methods may include having the patient deliver an aerosolized amount of surfactant through the patient's nostrils, and optionally repeating the delivery step a sufficient number of times until an effective amount of surfactant is delivered to the ET of a patient.

Other treatments for prophylaxis or the restoration of ear health similar to treatment 300 are also disclosed herein. These treatments may use surfactant compositions for otologic prophylaxis to reduce the frequency of, or to reduce the severity of the symptoms associated with the various pathologies and conditions described herein. For example, the treatment methods described herein may apply to OM with or without ear infection, hearing loss caused by middle ear fluid, speed and balance problems, behavior and sleep problems caused by recurrent or chronic OM. These treatment methods may also use surfactant compositions for otologic prophylaxis to reduce the frequency of extracranial complications associated with recurrent or chronic OM, wherein the extracranial complication include at least one of facial nerve paresis, labyrinthitis, mastoiditis, and petrositis. These treatment methods may also use surfactant compositions for otologic prophylaxis to reduce the frequency of, or to reduce the severity of symptoms related to, intracranial complications associated with recurrent or chronic OM, wherein the intracranial complications include at least one of brain abscess, epidural abscess, meningitis, lateral sinus thrombosis, subdural cavernous thrombosis, subdural abscess, cerebellitis, and labyrinth sclerosis. These treatment methods may also use surfactant compositions for otologic prophylaxis to reduce the risk or frequency of, or to reduce the severity of symptoms related to, cholesteatoma associated with recurrent or chronic OM. These treatment methods may also use surfactant compositions for otologic prophylaxis to reduce the frequency of, or reduce the severity of, damage to a patient's ear including scarring to the middle ear bones, damage to the eardrum, and damage to the inner ear. These treatment methods may also use surfactant compositions for otologic prophylaxis to reduce the frequency of, or reduce the severity of, dizziness, vertigo, nausea, or facial pain.

The reduction in the frequency or severity of the pathologies, symptoms, and conditions disclosed as a result of the disclosed treatments may vary depending on, for example, the patient, the dose, the frequency of the dosage, the age of the patient, the pathology, the symptom(s), and the condition. For example, in some patient populations, a treatment disclosed herein may reduce (e.g., by about 50%) the number of episodes of, or the pain over the course of a condition (e.g., OM) over a period of an episode or some other period of time (e.g., 1 year). For some symptoms the reduction in severity may be, for example, reduction of the infection time, improvement of hearing, reduction of fever, etc. Some treatments disclosed herein may reduce one or more of the pathologies, symptoms, or conditions disclosed herein by, for example, about 10%, about 20%, about 30%, about 40%, about 50%, about 60%, about 70%, about 80%, about 90% over the course of an episode. The reduced frequency resulting from prophylactic use of the compositions may be over a period of time, for example, of about 3 months, about 6 months, about 9 months, about 1 year, about 1.5 years, about 2 years, about 3 years, or more.

It is contemplated that the treatment methods described herein in addition to having prophylactic benefits may also benefit some patients by reducing the effects of one or more of the morbidities and/or symptoms described herein associated with the various diseases and conditions described herein.

According to an exemplary embodiment, the present disclosure relates to post-surgical treatment of a patient by the nasal delivery of the surfactant to inhibit ETD. A patient after having undergone surgery, for example, may have an increased risk of ETD, as well as a patient's profile according to one or more factors. The factors may include the type of surgery (if any), a patient's physiology, a patient's health, a patient's intended activities, or other factors. As part of the disclosed treatment, a caregiver may identify and assess one or more of the factors and then make a determination of whether the patient has an increased risk of ETD. If the caregiver determines that the patient has an increased risk of ETD then the caregiver may prescribe the nasal delivery of the surfactant. The determination by the caregiver may be made before or after surgery. In some embodiments, as part of the determination of the patient's increased risk, the caregiver may compare the patient's risk to a risk threshold, which makes administering the surfactant advisable considering various factors (e.g., cost, side effects, effectiveness, etc.). If the patient's risk exceeds the risk threshold, then the caregiver may prescribe the nasal delivery of the surfactant.

The type of surgery is one of the factors a caregiver may identify and assess for determining whether a patient has an increased risk of ETD. Certain surgeries may directly or indirectly affect the natural function of the ETs and in some cases may increase the risk of a patient developing ETD. For example, surgery of the ear, nose, or throat (i.e., surgeries proximate to the ETs) may increase the risk of developing ETD due to bleeding, inflammation, or infection. Such surgeries to the ear may include, for example, a myringoplasty, a tympanoplasty, an ossiculoplasty, a stapedectomy, a tympaneocentesis, a myringotomy, or other like surgery. Other surgeries to the nasal cavity, mouth, or throat may also increase the risk of developing ETD. Thus, the type of surgery the patient is undergoing is one factor that a caregiver may consider in determining and assessing whether a patient has an increased risk of developing ETD. In some cases, the type of surgery may be solely determinative of whether a patient is determined to have an increased risk of developing ETD post-surgery. In some embodiments, another factor a caregiver may identify and assess associated with the surgery is how successful the surgery was.

A patient's demographics, physiology, and/or medical history are other factors a caregiver may identify and assess for determining whether a patient has an increased risk of developing ETD. For example, a caregiver may consider a patient's age because of potential structural factors affecting risk of ETD dependent on a patient's age. For example, pediatric patients typically have shorter and narrower ETs, which make them more susceptible to blockage and developing ETD. A caregiver may also consider a patient's weight because an overweight or obese patient can be predisposed to ETD because of excess fatty deposits around the passageway of the ETs. A caregiver may also consider whether a patient has environmental allergies that may cause swelling around the ET openings, because swelling can restrict opening of the ETs and increase the risk of ETD. Whether a patient is a tobacco smoker or regularly exposed to tobacco smoke is another factor that a caregiver may consider because smoke can damage cilia (i.e., tiny hair-like cell extensions) in the ETs that create a current of mucus. A caregiver may also consider whether a patient has a history of ear infections or ETD. In some cases, a patient's demographics, physiology, and/or medical history may be solely determinative of whether a patient is determined to have an increased risk of developing ETD post-surgery.

A patient's health status or their status following surgery may also be identified and assessed by a caregiver for determining whether they have an increased risk of developing ETD. For example, if a patient has a compromised immune system for one or more reasons (e.g., the drug treatment proscribed in conjunction with the surgery), then they may have an increased risk of getting a cold or flu, which can increase the risk of ETD due to increased mucus, congestion, and inflammation in and around the ETs. In some cases, a patient's health (e.g., whether they are currently ill) may be solely determinative of whether a patient is determined to have an increased risk of developing ETD post-surgery.

In some cases, a caregiver may also consider a patient's planned activities when determining whether the patient has an increased risk of developing ETD. For example, if the patient is a frequent air traveler or planning to travel shortly after a surgery, the variability in the pressure caused by the elevation change may increase the risk of the patient developing ETD and the caregiver may consider this in making their determination. Scuba diving and driving in the mountains are other activities that expose a person and their ears to variability in pressure, which make increase the risk of developing ETD and may be planned activities a caregiver wants to consider. In some cases, a patient's planned activities may be solely determinative of whether a patient is determined to have an increased risk of developing ETD post-surgery.

A caregiver may identify and assess one or more of the factors when making a determination of whether a patient has an increased risk of developing ETD. In some cases, a combination of factors may be determinative of whether a patient is considered to have an increased risk of developing ETD post-surgery.

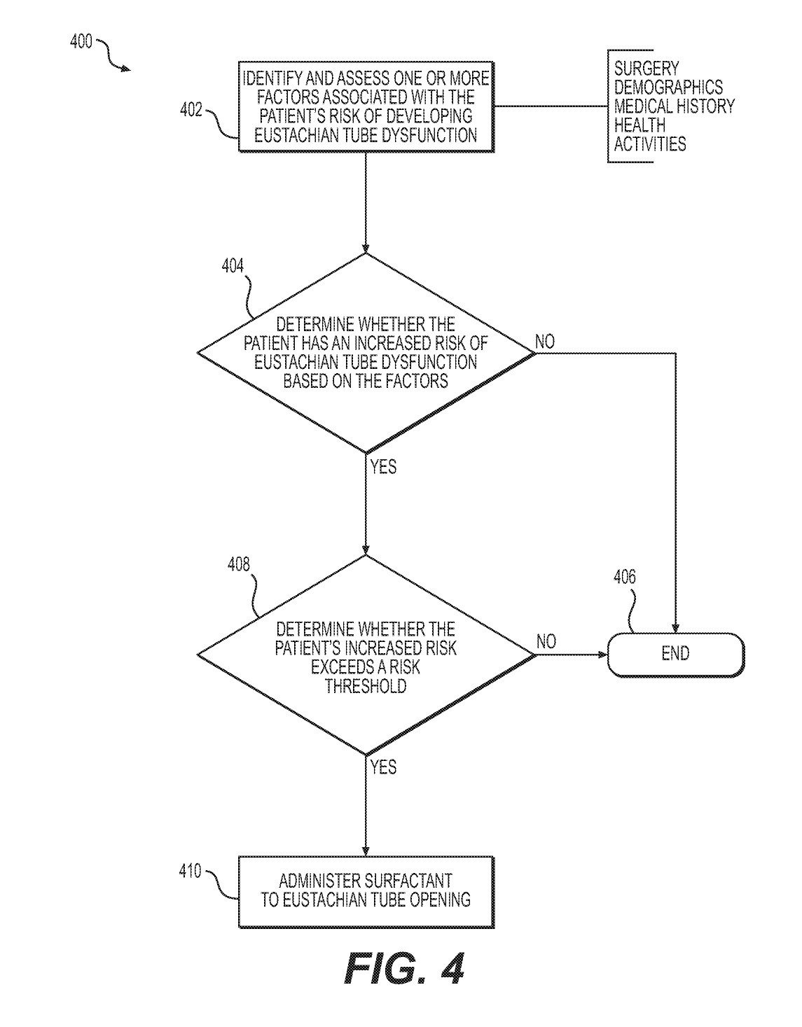

Turning to FIG. 4, a process flow schematic for treatment 400 inhibiting ETD is described. The treatment may include a caregiver identifying and assessing one or more factors associated with the patient's risk of developing ETD at step 402. The one or more factors may include, for example, the type of surgery, if any, the patient underwent (e.g., myringoplasty, a tympanoplasty, an oscciculoplasty, a stapedectomy, a tympaneocentesis, or a myringotomy), the patient's demographics (e.g., weight, age, etc.), the patient's medical history (e.g., allergies, previous ETD, smoker, etc.), the patient's health (e.g., compromised immune system), and the patient's planned activities (e.g., scuba diving, flying, etc.).

Next, based on the factors identified and assessed, a determination of whether the patient has an increased risk of ETD may be made at step 404. If it is determined that the patient does not have an increased risk of ETD (Step 404: No), then the treatment may end at step 406. If it is determined that the patient does have an increased risk of ETD (Step 404: Yes), then the caregiver can determine whether the patient's increased risk exceeds a risk threshold at step 408. A caregiver may utilize guidelines and/or prior experience in making the determination of whether the patient exceeds a risk threshold. According to an exemplary embodiment of the treatment, a mere increased risk may not make prescribing and administering the surfactant advisable. On the other hand, a recent history of ETD or middle-ear maladies may indicate surfactant treatment.

If is determined that the patient's increased risk does not exceed the risk threshold (Step 408: No), then the treatment may end at step 406. If it is determined that the patient's risk does exceed the risk threshold (Step 408: Yes), then the patient may be administered surfactant to the ETs, as described herein, at step 410. The treatment may also include optionally repeating the administering step a sufficient number of times until an effective amount of surfactant is delivered to the patient.

The methods of the present disclosure in addition to inhibiting ETD in a patient after surgery may also reduce healing time of the patient after the surgery. For example, a patient that has undergone a myringoplasty, a tympanoplasty, an oscciculoplasty, a stapedectomy, a tympaneocentesis, or a myringotomy may be able to speed up their healing after the surgery by administering the surfactant according to the methods described herein. In some embodiments, the primary purpose of the surfactant and treatment methods described herein may to reduce the healing time of the patient post-surgery.

In addition to inhibiting ETD in a patient after surgery, the surfactant and methods of the present disclosure may also help stabilize the middle ear space over time.

### CLINICAL EXAMPLES

Eight persons (cases 1-8 below) were exposed to a composition of DPPC:CP [16:1 w/w] in HFA-134a, with each 0.1 mL spray delivering 2.5 mg of active ingredient), and 1 other person (case 9) was exposed to a composition of DPPC:CP [200:1 w/w] in HFA-134a, with each 0.1 mL spray delivering 5 mg of active ingredient.

Case 1: Caucasian male, 54 years of age, 2 sprays of the composition per each nostril twice daily for 10 days (total exposure 20 mg per day×10 days=200 mg) used for “ear blockage sensation”. A “cool” sensation in nostril with administration was reported. There was no report of burning sensation, noxious odor, discomfort, or epistaxis with administration. Ear symptoms were cleared.

Case 2: Asian-American female, 48-50 years of age, 2 sprays of the composition per each nostril during airplane flights when experiencing “squeeze” negative pressure sensation in ears. Applied a total of 10 times on various flights (total exposure estimated to be 100 mg). A mild “cool” sensation in nostril with administration was reported. There was no report of burning sensation, noxious odor, discomfort, or epistaxis with administration. The treatment was reported to have cleared “squeeze” in under 10 minutes each time it was used, and that effect lasted for the duration of the flight (including some flights that were 5-6 hours long).

Case 3: Caucasian male, 42 years of age. Severe ET symptoms manifested as ear pain with airplane flights for subject's “entire life”. Two sprays of composition per each nostril (each dose 10 mg) before takeoff and/or landing on several cross Atlantic air flights. Unable to estimate total exposure. No adverse events were reported. There was no report of cool or burning sensation, noxious odor, epistaxis, or nasal pain with administration. No ear pain occurred at all with air flights when the spray was used.

Case 4: Caucasian male, 50 years of age, 2 sprays of composition per each nostril once (total exposure 10 mg) for left ET blockage due to acute rhinitis. No adverse events were reported. There was no report of burning sensation, noxious odor, discomfort, epistaxis, or cool sensation with administration. Video documentation made of ET status before and at intervals following intranasal administration shows onset of effect in opening ET within 10 minutes, with return to normal in 20 minutes. Further application was not needed or done.

Case 5: Asian-American female, 4 years of age. AOM with severe ear pain and bulging tympanic membrane. The child initially received a total of 2 sprays of composition in each nostril twice daily for a total of 3 doses (total exposure 30 mg). No adverse events were reported. No pain or odd sensation in nose with application was reported. No epistaxis occurred. Resolution of pain began within 5 minutes of composition application and was complete within 10 minutes. Child came willingly for further applications. There was no need for systemic analgesics or oral or topical antibiotics.

Case 6: Caucasian male, 48 years of age. Two sprays of composition per each nostril administered once (total exposure 10 mg). Treatment given for video-documentation of ET action in a normal, healthy adult. A slight “cool” sensation in nostril with administration was reported. There was no report of burning sensation, noxious odor, discomfort, or epistaxis with administration. Video shows opening of ET several minutes after application.

Case 7: Caucasian female, 21 years of age. Chronic serous OM in both ears with conductive hearing loss due to upper respiratory infection and airplane flights. Symptoms were present for over 1 month, despite oral and intranasal steroid administration and antibiotics. She was offered an in-office myringotomy procedure, but was very scared of needle/manipulation. Two sprays of composition were administered in each nostril in the office (10 mg). At 10 minutes post-treatment, her ears started “popping”—first time in a month. At 20 minutes post-treatment, she felt like her ears were back to normal. Examination, tympanogram and audiogram confirmed this improvement—return to normal. She used the composition twice daily for the next 3 days and follow-up examination at that time showed normal ears and hearing, and normal nasal examination. Total exposure was 70 mg. No adverse events were reported. She denied “cool” sensation, burning, irritation, noxious odor, or epistaxis with administration. She also denied having even temporary recurrence of her previously month-long ear symptoms during the 3 days and afterwards.

Case 8: Asian-American male, 75 years of age. He has a history of frequent, recurrent AOM intermittently since childhood. As a result, he has significant sensorineural hearing loss. During a flight overseas he developed right serous OM (fluid behind the tympanic membrane with reduced hearing). He treated himself with oral CLARITIN-D (loratadine and pseudoephedrine) and intranasal AFRIN (oxymetazoline). At his overseas destination, he saw an ENT physician who performed a myringotomy on his right tympanic membrane. His ear pressure improved after the procedure; no ear fluid was noted. He continued treatment with oral CLARITIN-D. Within a couple of months after the overseas trip, on at least two occasions immediately prior to flying, he used 2 sprays in each nostril (10 mg) of the composition (2.5 mg/spray, 16:1 ratio), along with the oral CLARITIN-D. He did not report having any ear problems on these flights. He also did not report any adverse events after using the product.

Case 9: Caucasian male, 68 year of age. Chronic ETD manifested as middle ear ache whenever flying in an airplane. He used the investigational product (estimated as 20 times over a 5-year period) 5 to 10 minutes prior to takeoff and reported that he no longer had issues with his middle ears when flying. The dose he used was 2 sprays in each nostril of the 200:1 w/w formulation (each spray 5 mg) for a total dose 20 mg per treatment. There was no report of burning sensation, noxious odor, discomfort, or epistaxis with administration.

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims. The terms and expressions which have been employed in the foregoing specification are used therein as terms of description and not limitation, and there is no intention, in the use of such terms and expressions, of excluding equivalents of the features shown and described or portions thereof, it being recognized that the scope of the disclosure is defined and limited only by the following claims.

What is claimed is:

1. A prophylactic method for preventing otitis media in a patient comprising administration of a prophylactically effective dosage of a surfactant composition through the nostrils of the patient.

2. The method according to claim 1, further comprising a plurality of administrations of surfactant composition over time.

3. The method of claim 1, wherein the composition prevents chronic otitis media with effusion.

4. The method of claim 1, wherein the composition prevents recurrent episodes of otitis media.

5. The method of claim 1, wherein the composition reduces the severity of an otitis media episode.

6. The method of claim 1, wherein the patient is not administered a systemic antibiotic for the treatment of otitis media.

7. The method of claim 1, wherein the patient does not undergo a myringotomy or tympanostomy tube insertion.

8. The method of claim 1, wherein said administration further comprises dispersing said surfactant onto a region of the nasopharynx containing the Eustachian tube opening.

9. The method of claim 1, wherein the patient is prone to baro-challenge induced Eustachian tube dysfunction.

10. The method of claim 1, wherein the surfactant composition is a liquid.

11. The method of claim 1, wherein the surfactant composition is a powder.

12. The method of claim 1, wherein the surfactant composition comprises DPPC.

13. A prophylactic method for reducing the likelihood of Eustachian tube dysfunction in a patient, comprising administration of a prophylactically effective dosage of a surfactant composition through the nostrils of the patient.

14. The method according to claim 11, further comprising a plurality of administrations of surfactant composition over time.

15. The method of claim 13, wherein the patient has a history of Eustachian tube dysfunction.

16. The method of claim 13, wherein the patient is not administered an antibiotic.

17. The method of claim 13, wherein said administration further comprises dispersing said surfactant onto a region of the nasopharynx containing the Eustachian tube opening.

18. The method of claim 13, wherein the surfactant composition is administered to the patient before the patient is subject to an ambient pressure change.

19. The method of claim 13, wherein the surfactant composition is a liquid.

20. The method of claim 13, wherein the surfactant composition is a powder.

21. The method of claim 13, wherein the surfactant composition comprises DPPC.

22. A method for reducing the severity of symptoms of otitis media before onset in a patient comprising administration of a prophylactically effective dosage of a surfactant composition through the nostrils of the patient.

23. The method according to claim 22, further comprising a plurality of administrations of surfactant composition over time.

24. The method of claim 22, wherein the patient is a neonate.

25. The method of claim 22, wherein said administration further comprises dispersing said surfactant onto a region of the nasopharynx containing the Eustachian tube opening.

26. The method of claim 22, wherein the patient is prone to baro-challenge induced Eustachian tube dysfunction.

27. A prophylactic method for preventing baro-challenge Eustachian tube dysfunction comprising administration of a prophylactically effective dosage of a surfactant composition through the nostrils of the patient before an ambient pressure change.

28. The method according to claim 27, further comprising a plurality of administrations of surfactant composition over time.

29. The method of claim 27, wherein the composition reduces the severity of an otitis media episode.

30. The method of claim 27, wherein said administration further comprises dispersing said surfactant onto a region of the nasopharynx containing the Eustachian tube opening.
